(19) United States
(12) Patent Application Publication
(10) Pub. No.: US 2025/0224853 A1
(43) Pub. Date: Jul. 10, 2025

(54) DIGITAL ASSISTANCE SYSTEM, METHOD AND DEVICE

(71) Applicant: Måna Care AB, Stallarholmen (SE)

(72) Inventors: Jeffrey DeAngelis, Mountain View, CA (US); Anders Reisch, Stockholm (SE)

(21) Appl. No.: 18/999,265

(22) Filed: Dec. 23, 2024

Related U.S. Application Data
(60) Provisional application No. 63/618,187, filed on Jan. 5, 2024.

(30) Foreign Application Priority Data
Jul. 12, 2024 (EP) 24188355

(51) Int. Cl. G06F 3/0482; A61B 5/01; A61B 5/024; A61B 5/145; G06F 3/041; G06F 3/04845; G16H 80/00

(52) U.S. Cl. CPC G06F 3/0482; A61B 5/01; A61B 5/024; A61B 5/14542; G06F 3/0412; G06F 3/04845; G16H 80/00

(57) ABSTRACT

A digital assistance system, method and device for assisting users. The digital assistance system includes an assistance device having a finger touch pad and sensors operably coupled with the finger touch pad and configured to measure at least one of a heart rate, a blood-oxygen level and a temperature of the user. The digital assistance device is also able to include a communication application for providing non-character based format media data communication via a Message Queueing Telemetry Transport (MQTT) communication protocol.

### RELATED APPLICATIONS

This application claims priority under 35 U.S.C. § 119(e) of U.S. Provisional Patent Application Ser. No. 63/618, 187, filed Jan. 5, 2024, and entitled “INSPIRING DIGITAL INCLUSION BY SIMPLIFYING CARE,” and of European Patent Application No. 24188355, filed Jul. 12, 2024, and entitled “A COMMUNICATIONS ARRANGEMENT AND METHOD, AND A COMPUTER PROGRAM PRODUCT FOR PERFORMING THE METHOD,” both of which are hereby incorporated by reference in their entirety.

### FIELD OF THE INVENTION

The present invention relates to the field of digital assistance. Specifically, the present invention relates to a system for enabling publish and subscribe communication between multiple devices.

### BACKGROUND OF THE INVENTION

Caregiving platforms are increasingly needed to provide support and care, in particular to a growing elderly population. According to the United Nations 2019 study of the aging world population, the growing elderly population gap (65+ year olds) vs. the rest of the adult population is expected to grow to a 40:1 ratio by 2030 in the United States and Europe and a ratio of 20:1 on a worldwide basis. However, the typical single-touch fixed in-home visit is inadequate for keeping up with these caregiving demands. In particular, without daily and throughout the day care, it is difficult to ensure performance of important daily activities such as taking medicine, eating meals, dressing appropriately for the current temperature/weather/season and/or other type of activities.

Digital caregiving platforms are able to lessen some of these issues, but create new issues relating to user interfaces as well as communication reliability, speeds and/or response times. In particular, the graphical user interfaces (GUIs) of digital caregiving platforms can be difficult for elderly users to effectively manipulate due to digital icon/button sizes being too small and difficult to press, text/images difficult to read/view, and audio too quiet and difficult to understand. Further, caregiving platforms are often limited to software downloaded/operating on generic computing devices. Thus, there is often a problem of the devices operating the caregiving platforms being unable to perform health check measurements/procedures on clients, which instead requires clients to be seen in person by a healthcare professional and prevents daily checkups and/or daily monitoring of client health data.

Additionally, digital caregiving platforms are often unable to handle situations that require prompt responses. For example, it may be crucial that an alarm is responded to as quickly as possible. For someone who is cognitively impaired, real-time or near real-time response may be very important. Current digital caregiving platforms typically use a hypertext transfer protocol (HTTP)-based communication protocol. HTTP is commonly used for all types of communication and has the advantage of being widely compatible. However, HTTP includes technical problems (in particular in the caregiving field) in that:
HTTP only responds to data requests (uni-directional data flow);
HTTP requires a lot of overhead, increasing the bandwidth requirement;
HTTP requires the user to open and close a connection each time a data packet is sent; and
HTTP scalability is bandwidth limited.
   Each of these properties make HTTP less suitable for applications that require real-time connectivity (e.g. digital caregiving). As a result, traditional digital caregiving platforms using HTTP are unable to provide the technical features required for efficient data communication. Other protocols such as MQTT, while being able to respond more quickly, have the problem of being unable to transmit all the different formats of media data (e.g. data having a non-character based formats such as .mp3,.wav, .mp4, .json, .png, .jpg, etc.). Thus, such a protocol is unable to handle the multiple different types of media data formats and provide video and/or audio data between devices which is often necessary in a caregiving and other environments.

### SUMMARY OF THE INVENTION

A digital assistance system, method and device is able to includes a device (e.g. assistance device) having a finger touch pad and sensors operably coupled with the finger touch pad and configured to measure at least one of a heart rate, a blood-oxygen level and a temperature of the user. The device is also able to include a communication application for providing non-American Standard Code for Information Interchange (ASCII) format media data communication via a Message Queueing Telemetry Transport (MQTT) communication protocol.

A first aspect is directed to an assistance device for providing care to one or more users. The assistance device comprises a protective housing, a touch screen coupled with the housing for displaying images and receiving touch commands, one or more cameras coupled with the housing, one or more microphones coupled with the housing, one or more speakers coupled with the housing, a finger touch pad coupled with the housing for receiving contact from a finger of a user, one or more sensors operably coupled with the finger touch pad and configured to measure at least one of a heart rate, a blood-oxygen level and a temperature of the user based on measurements received by the sensors via the finger touch pad while being contacted by the finger of the user and a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device.

In some embodiments, wherein upon selection of one of the digital activity buttons, the application publishes a message that is received by one or more trusted advisor devices that are subscribed to the assistance device, the message identifying the activity as being completed. In some embodiments, upon receiving a fullscreen command, the application is configured to change the image/video window from a first size that is smaller than the touch screen to a larger size that is the same size as the touch screen thereby increasing the size of images or video displayed by the image/video window. In some embodiments, the graphical user interface includes a weather tile and the assistance application includes a generative artificial intelligence weather agent that generates images and/or text on the touch screen indicating current suggested clothing and future suggested clothing based on at least one of a current temperature, a current weather, a current humidity and a forecast weather for a location of the assistance device. In some embodiments, the graphical user interface further comprises a digital call button that is associated with one or more trusted advisor devices, and upon receiving a call command, the application is configured to initiate a telephone call to at least one of the trusted advisor devices. In some embodiments, the graphical user interface further comprises a digital assistant button associated with a database of personal data about a life of the user, and upon receiving a personal query about the user, a generative artificial intelligence memory agent of the application outputs an answer to the personal query based on the personal data of the user.

In some embodiments, the graphical user interface further comprises a digital next activities button, and upon receiving a selection of the digital next activities button, the application is configured to display a list of upcoming activities associated with the user. In some embodiments, the application comprises a communication module, and upon receipt of media data having a non-character based format, the communication module performs a binary encoding on the media data to convert the media data into one or more corresponding characters of a character based format, adds the corresponding characters as payload data to one or more MQTT messages and transmits the MQTT messages to a trusted advisor device according to a MQTT protocol. In some embodiments, upon receipt of an MQTT message from the trusted advisor device, the communication module parses the payload data from the MQTT message, and converts characters of the payload data to corresponding the media data having the non-character based format according to the binary encoding, and further wherein the application outputs the media data on at least one of the touchscreen and the speakers. In some embodiments, the media data comprises at least one of images, video and audio. In some embodiments, the binary encoding is Base64 encoding.

A second aspect is directed to an assistance system for providing care to one or more users. The assistance system comprises an assistance device including a protective housing, a touch screen coupled with the housing for displaying images and receiving touch commands, one or more cameras coupled with the housing, one or more microphones coupled with the housing, one or more speakers coupled with the housing, a finger touch pad coupled with the housing for receiving contact from a finger of a user, one or more sensors operably coupled with the finger touch pad and configured to measure at least one of a heart rate, a blood-oxygen level and a temperature of the user based on measurements received by the sensors via the finger touch pad while being contacted by the finger of the user and a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device and one or more trusted advisor devices including a trusted advisor application for communicating with the assistance device.

In some embodiments, wherein upon selection of one of the digital activity buttons, the assistance application publishes a message that is received by one or more trusted advisor devices that are subscribed to the assistance device, the message identifying the activity as being completed. In some embodiments, upon receiving a fullscreen command, the assistance application is configured to change the image/video window from a first size that is smaller than the touch screen to a larger size that is the same size as the touch screen thereby increasing the size of images or video displayed by the image/video window. In some embodiments, the graphical user interface includes a weather tile and the assistance application includes a generative artificial intelligence weather agent that generates images and/or text on the touch screen indicating current suggested clothing and future suggested clothing based on at least one of a current temperature, a current weather, a current humidity and a forecast weather for a location of the assistance device. In some embodiments, the graphical user interface further comprises a digital call button that is associated with one or more trusted advisor devices, and upon receiving a call command, the application is configured to initiate a telephone call to at least one of the trusted advisor devices. In some embodiments, the graphical user interface further comprises a digital assistant button associated with a database of personal data about a life of the user, and upon receiving a personal query about the user, a generative artificial intelligence memory agent of the application outputs an answer to the personal query based on the personal data of the user.

In some embodiments, the graphical user interface further comprises a digital next activities button, and upon receiving a selection of the digital next activities button, the assistance application is configured to display a list of upcoming activities associated with the user. In some embodiments, the assistance application comprises a communication module, and upon receipt of media data having a non-character based format, the communication module performs a binary encoding on the media data to convert the media data into one or more corresponding characters of a character based format, adds the corresponding characters as payload data to one or more MQTT messages and transmits the MQTT messages to a trusted advisor device according to a MQTT protocol. In some embodiments, upon receipt of an MQTT message from the trusted advisor device, the communication module parses the payload data from the MQTT message, and converts characters of the payload data to corresponding the media data having the non-character based format according to the binary encoding, and further wherein the application outputs the media data on at least one of the touchscreen and the speakers. In some embodiments, the media data comprises at least one of images, video and audio. In some embodiments, the binary encoding is Base64 encoding.

Another aspect is directed to a method for providing care to one or more users. The method comprises providing an assistance device including, a protective housing, a touch screen coupled with the housing for displaying images and receiving touch commands, one or more cameras coupled with the housing, one or more microphones coupled with the housing, one or more speakers coupled with the housing, a finger touch pad coupled with the housing for receiving contact from a finger of a user, one or more sensors operably coupled with the finger touch pad and a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device and measuring, with the sensors, at least one of a heart rate, a blood-oxygen level and a temperature of the user based on measurements received by the sensors via the finger touch pad while being contacted by the finger of the user.

In some embodiments, the method further comprises, upon selection of one of the digital activity buttons, publishing a message with the application to one or more trusted advisor devices that are subscribed to the assistance device, the message identifying the activity as being completed. In some embodiments, the method further comprises, upon receiving a fullscreen command, changing the image/video window from a first size that is smaller than the touch screen to a larger size that is the same size as the touch screen with the application thereby increasing the size of images or video displayed by the image/video window. In some embodiments, the graphical user interface includes a weather tile, the method further comprising generating images and/or text on the touch screen with a generative artificial intelligence weather agent of the assistance application, the images and/or text indicating current suggested clothing and future suggested clothing based on at least one of a current temperature, a current weather, a current humidity and a forecast weather for a location of the assistance device. In some embodiments, the graphical user interface further comprises a digital call button that is associated with one or more trusted advisor devices, the method further comprising, upon receiving a call command, initiating a telephone call to at least one of the trusted advisor devices. In some embodiments, the graphical user interface further comprises a digital assistant button associated with a database of personal data about a life of the user, the method further comprising, upon receiving a personal query about the user, outputting an answer to the personal query based on the personal data of the user with a generative artificial intelligence memory agent of the application.

In some embodiments, the graphical user interface further comprises a digital next activities button, the method further comprising, upon receiving a selection of the digital next activities button, displaying a list of upcoming activities associated with the user. In some embodiments, the assistance application comprises a communication module, the method further comprising, with the communication module and upon receipt of media data having a non-character based format, performing a binary encoding on the media data to convert the media data into one or more corresponding characters of a character based format, adding the corresponding characters as payload data to one or more MQTT messages and transmitting the MQTT messages to a trusted advisor device according to a MQTT protocol. In some embodiments, the method further comprises, with the communication module and upon receipt of an MQTT message from the trusted advisor device, parsing the payload data from the MQTT message, converting characters of the payload data to corresponding the media data having the non-character based format according to the binary encoding, and outputting the media data on at least one of the touchscreen and the speakers with the application. In some embodiments, the media data comprises at least one of images, video and audio. In some embodiments, the binary encoding is Base64 encoding.

Another aspect is directed to a device for providing a Message Queueing Telemetry Transport (MQTT) communication program to one or more users. The device comprises an output interface for outputting first media data from the device, an input interface for inputting media data having the varied non-character-based formats and a non-transitory computer-readable memory storing a communication application, wherein upon receipt of the media data having the varied non-character-based formats, the communication application standardizes the non-character-based format media data into a character-based format by converting each chunk of the non-character-based format media data to a character of the character-based format, adds the characters as payload data to one or more MQTT messages and transmits the MQTT messages to a second device according to MQTT protocol.

In some embodiments, upon receipt of another MQTT message from the second device, the communication application converts each of the characters of the payload data of the another MQTT message to a corresponding chunk of non-character-based format media data, and outputs the corresponding chunks of the non-character-based format media data via the output interface. In some embodiments, the non-character-based format media data comprises at least one of images, video and audio. In some embodiments, the character-based format is ASCII and the converting of each chunk of the non-character-based format media data to the character of the character-based format comprises performing a Base64 encoding the non-character-based format media data. In some embodiments, the output interface comprises one or more of a display, a touchscreen and speakers. In some embodiments, the input interface comprises one or more of a camera, a microphone, a touchscreen, a keyboard, a mouse, a graphical user interface and a network interface.

Another aspect is directed to a system for providing a Message Queueing Telemetry Transport (MQTT) communication program to one or more users. The system comprises a first device including a first output interface for outputting first media content, a first input interface for inputting first non-character-based format media data, and a first non-transitory computer-readable memory storing a first communication application and a second device including a second output interface for outputting second media content, a second input interface for inputting second non-character-based format media data, and a second non-transitory computer-readable memory storing a second communication application, wherein upon input of the first non-character-based format media data via the first input interface, the first communication application standardizes the first non-character-based format media data into a character-based format by converting each chunk of the first non-character-based format media data to a character of the character-based format, adds the characters as payload data to one or more first MQTT messages and transmits the first MQTT messages to the second device according to MQTT protocol.

In some embodiments, upon receipt of at least one of the first MQTT messages from the first device, the second communication application converts each of the characters of the payload data of the at least one of the MQTT messages to a corresponding chunk of the first non-character-based format media data, and outputs the corresponding chunks of the non-character-based format media data via the second output interface. In some embodiments, the first non-character-based format media data comprises at least one of images, video and audio. In some embodiments, the character-based format is ASCII and the converting of each chunk of the first non-character-based format media data to the character of the character-based format comprises performing a Base64 encoding the first non-character-based format media data. In some embodiments, the first output interface comprises one or more of a display, a touchscreen and speakers. In some embodiments, the first input interface comprises one or more of a camera, a microphone, a touchscreen, a keyboard, a mouse, a graphical user interface and a network interface.

Another aspect is directed to a method for providing a Message Queueing Telemetry Transport (MQTT) communication program to one or more users. The method comprises providing an MQTT communication device including an output interface for outputting first media data from the device, an input interface for inputting media data having the varied non-character-based formats and a non-transitory computer-readable memory storing a communication application, upon receipt of the media data having the varied non-character-based formats by the communication application, standardizing the non-character-based format media data into a character-based format by converting each chunk of the non-character-based format media data to a character of the character-based format, adding the characters as payload data to one or more MQTT messages with the communication application and transmitting the MQTT messages to a second device according to MQTT protocol with the communication application.

In some embodiments, the method further comprises upon receipt of another MQTT message from the second device, converting each of the characters of the payload data of the another MQTT message to a corresponding chunk of non-character-based format media data with the communication application and outputting the corresponding chunks of the non-character-based format media data via the output interface with the communication application. In some embodiments, the non-character-based format media data comprises at least one of images, video and audio. In some embodiments, the character-based format is ASCII and the converting of each chunk of the non-character-based format media data to the character of the character-based format comprises performing a Base64 encoding the non-character-based format media data. In some embodiments, the output interface comprises one or more of a display, a touchscreen and speakers. In some embodiments, the input interface comprises one or more of a camera, a microphone, a touchscreen, a keyboard, a mouse, a graphical user interface and a network interface.

### BRIEF DESCRIPTION OF THE DRAWINGS

FIG. 1 illustrates a digital assistance system according to some embodiments.

FIG. 2 illustrates a client device according to some embodiments.

FIG. 3 illustrates a block diagram of an exemplary advisor device according to some embodiments.

FIG. 4 illustrates an exemplary screenshot of the graphical user interface as displayed on the touchscreen according to some embodiments.

FIG. 5 illustrates an exemplary screenshot of the graphical user interface as displayed on an advisor device according to some embodiments.

FIG. 6 illustrates a method of providing care to one or more users with client device including a digital assistance program according to some embodiments.

FIG. 7 illustrates a method for providing a modified MQTT communication program to one or more users according to some embodiments.

FIG. 8 illustrates an exemplary communication module data conversion process according to some embodiments.

### DETAILED DESCRIPTION OF THE INVENTION

Embodiments described herein disclose a digital assistance system, method and device for providing care to users. The digital assistance system is able to include a dedicated assistance device having a built-in finger touch pad and sensors operably coupled with the finger touch pad and configured to measure at least one of a heart rate, a blood-oxygen level and a temperature of the user. As a result, the digital assistance device is able to solve the problem of digital assistance systems not being able to measure accurate health data on demand, periodically and/or upon request by a remote caregiver. Specifically, unlike prior generic devices, the dedicated assistance device having a built-in touch pad and corresponding sensors/software for checking health data provides the ability of users to routinely update their health data without meeting with a caregiver in person.

The assistance device is also able to include a communication application for providing non-character based format (e.g. American Standard Code for Information Interchange (ASCII)) media data communication via a Message Queueing Telemetry Transport (MQTT) communication protocol. As a result, the system provides the advantage of solving the problem of inadequate assistance GUIs with small icons, text and/or quiet audio that is unable to be effectively used by elderly users with poor eyesight hearing and/or dexterity. Additionally, the system solves the problem of HTTP based systems that can only respond to data requests (uni-directional data flow), require a lot of overhead, increasing the bandwidth requirements, require the user to open and close a connection each time a data packet is sent, and have bandwidth limited scalability, by converting non-character based format media data (e.g. audio, video, images, etc.) to an character based format (e.g. ASCII) that is able to be transmitted via MQTT protocol. Moreover, the system is able to provide the benefit of helping trusted advisors (e.g. professional caregivers) care for their clients partly remotely, through streamlining administration tasks to communicate the well being, observations, and needs of their clients with family members and medical support teams.

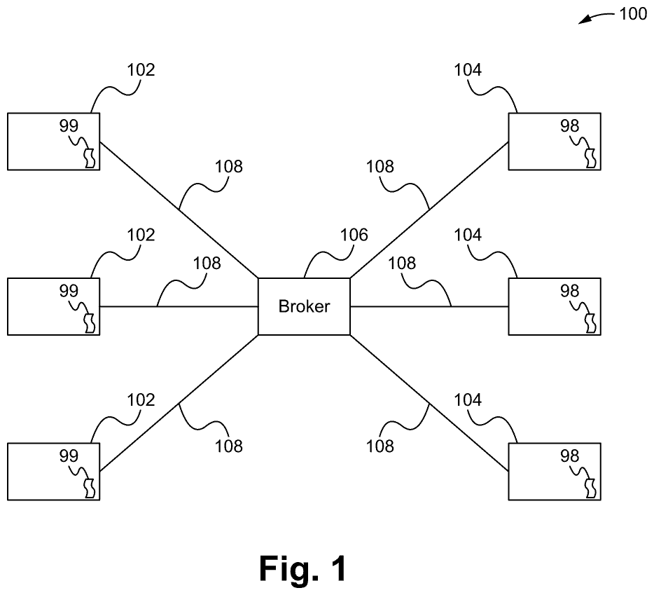

FIG. 1 illustrates a digital assistance system 100 according to some embodiments. As shown in FIG. 1, the system 100 comprises one or more digital assistance client devices 102 each including a digital assistance program 99, one or more advisor devices 104 each including a an advisor program 98, and one or more broker devices 106. The client devices 102 and the advisor devices 104 are each coupled with the broker devices 106 via one or more networks 108. The networks 108 are able to be one or a combination of wired or wireless networks as are well known in the art. Although as shown in FIG. 1, three client devices 102 and three advisor devices 104 are coupled with a single broker device 106, it is understood that the system 100 is able to comprise any number of client devices 102, advisor devices 104 and/or broker devices 106 coupled together via the networks 108. The networks 108 are able to comprise a cellular data network, a WiFi network and/or other types of networks or a combination thereof.

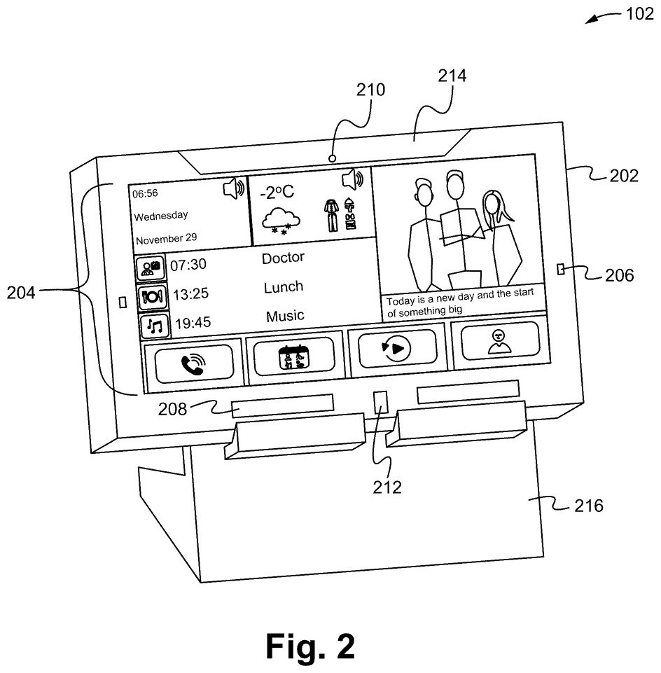

FIG. 2 illustrates a client device 102 according to some embodiments. As shown in FIG. 2, the client device 102 comprises a housing 202, a touchscreen/display 204, one or more microphones 206, one or more speakers 208, one or more cameras 210, one or more sensors/sensor pad 212 and a light/visual alarm 214 all coupled together within or partially within the housing 202. In some embodiments, the housing 202 is able to be detachably coupled to a support structure 216 having a slot for receiving the housing 202. Specifically, the slot is able to include a window such that the sensor pad 212 is uncovered even when the housing 202 is coupled to the support structure 216. The sensors and/or sensor pad 212 are able to be configured to input/measure health check-up data comprising: user body temperature data; heart rhythm data (e.g. to check for atrial fibrillation); blood-oxygen data (e.g. via a pulse oximeter or SpO2 sensor that measures an amount of oxygen in the blood of the user based on how much light passes through a finger pressed against the sensor pad 212); heart rate data (e.g. pulse rate based on small changes in blood volume within user's arteries caused by user's heartbeat indicated by reflections of light waves emitted into the skin at the sensor pad 212); and/or blood pressure data (e.g. pulse transit time via electrocardiogram sensor or optical heart rate sensor). As a result, client device 102 provides an advantage over generic devices by enabling periodic and/or on demand health data measurements/updates via its sensor pad and associated sensors 212 along with its support structure 216 having a window for facilitating easy use of the sensor pad 212 (even when the device 102 is coupled with the support structure 216). In some embodiments, the housing 202 and/or device 102 is 10 to 14 inches in width, 7 to 11 inches in height and 0.5 to 1.5 inches in depth (e.g. 14×9.2×1 inch). Alternatively, one or more of the dimensions of the housing 202 and/or device 102 are able to be larger or smaller. For example, in some embodiments the housing 202 and/or device 102 have a width and/or length substantially similar to that of a credit card (e.g. 3.375 inches wide by 2.125 tall).

Although not shown in FIG. 2, the client device 102 is able to also comprise a power source (e.g. batteries, plugs, or combination thereof), one or more memories (e.g. non-transitory computer-readable media), one or more processors and/or one or more input/output interfaces operatively coupled together to form a processing network. In particular, the digital assistance program 99 is able to be stored on the memories and executed by the processors of the processing network such that the processing network (which is able to be operably coupled with one or more of the touchscreen/display 204, the microphones 206, the speakers 208, the cameras 210, the sensors/sensor pad 212 and the light/visual alarm 214) is able to perform the functions of the client device/assistance program 102, 99 described herein. Alternatively, one or more of the touchscreen/display 204, the microphones 206, the speakers 208, the cameras 210, the sensors/sensor pad 212, the light/visual alarm 214 and/or the support structure 216 are able to be omitted from the device 102.

In some embodiments, the entirety of the digital assistance program 99 is able to be stored and provided by the digital assistance client 102. Alternatively, some of the data for providing certain functions of the application 99 is able to be stored in one or more remote databases (e.g. third party databases) such that the application 99 must connect to the remote databases in order to provide the functionality. For example, weather data is able to be downloaded from a remote database in order for the application 99 to provide the weather display function.

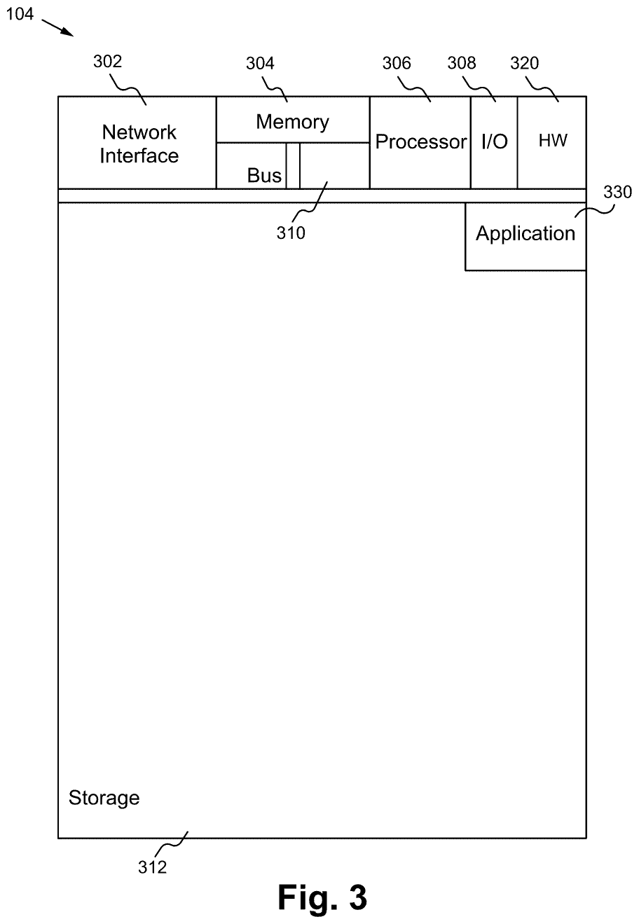

FIG. 3 illustrates a block diagram of an exemplary advisor device 104 according to some embodiments. As shown in FIG. 3, the advisor device 104 is able to comprise a network interface 302, a memory 304, a processor 306, I/O device(s) 308, a bus 310, a storage device 312, software 330 and hardware 320. Alternatively, one or more of the illustrated components are able to be removed or substituted for other components well known in the art. The storage device 312 is able to include a hard drive, solid state storage, network-attached storage, cloud storage, RAM, SRAM, CDROM, CDRW, DVD, DVDRW, flash memory card or any other storage device. The network interface 302 is able to comprise a network card connected to a WAN such as Cellular LTE or other types of LANs such as Ethernet or WLAN. The I/O device(s) 308 are able to include one or more of the following devices capable of inputting or conveying data such as a keyboard, mouse, monitor, display, printer, scanner, modem, touchscreen, button interface, speech recognition interface, and other devices. The advisor program 98 is able to be stored in the storage device 312 and/or memory 304 and processed by the processor 306 during operation. Additionally, it should be noted that it is contemplated that some or all of the features/components of the advisor devices 104 described herein are able to be included in the client devices 102, and/or some or all of the features/components of the client devices 102 described herein are able to be included in the advisor devices 104.

In some embodiments, the entirety of the dashboard program 98 is able to be stored and provided by the advisor device 104. Alternatively, some of the data for providing certain functions of the application 98 is able to be stored in one or more remote databases (e.g. third party databases) such that the application 98 must connect to the remote databases in order to provide the functionality.

### The Digital Assistance Program

The digital assistance program 99 is able to comprise a login and registration module, a translation module, a calendar module, a weather module, an activities module, a call module, a next activities module, a messages module, a digital assistant module, a speech to text module, a display module, a sensor module and a communication module. In some embodiments, the program 99 is able to comprise a fall detection module configured to detect when the device 102 and/or the user holding the device 102 is dropped. In such embodiments, the fall detection module is able to replace the sensor module and/or sensor pad 212. Alternatively, one or more of the modules are able to be omitted and/or combined into a single module. The functions of one or more of the modules are accessible to users via a graphical user interface displayed on the touchscreen 204. For example, the modules are able to receive input via the graphical user interface (as well as via the microphone 206, camera 210, sensor pad 212 and/or network 108) and perform functions described herein based on the input (including providing output via one or more of the speakers 208, light 214, display 204 and/or network 108).

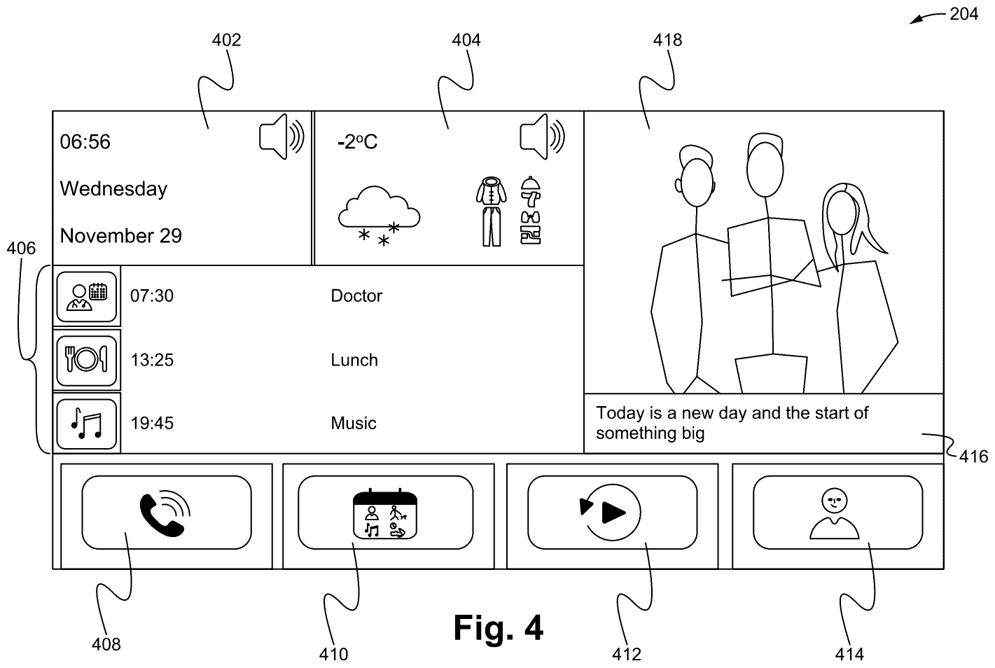

FIG. 4 illustrates an exemplary screenshot of the graphical user interface as displayed on the touchscreen 204 according to some embodiments. As shown in FIG. 4, the graphical user interface includes a calendar tile 402, a weather tile 404, one or more digital activity buttons 406 (each having an associated label and time), a digital call button 408, a digital next activities button 410, a digital messages button 412, a digital assistant button 414, a speech to text box 416 and a display window 418. Alternatively, one or more of the components of the graphical user interface shown in FIG. 4 are able to be omitted. Additionally, the relative size and/or position of each of the components is able to be dynamically adjusted. For example, the display window 418 is able to be enlarged to be full screen and cover the entire display 204 for a period of time.

The login and registration module enables a user to create an account for the program 99 by inputting identification/contact information (e.g. username, password information, an email, passcodes, alternate contact methods, biometrics, two-factor authentication using text, voice or email and/or a security token) via the graphical user interface that is then associated with the account such that the identification/contact information is able to be used to identify the user when logging onto the program. The identification/contact information associated with the account is able to be stored in an account database. After an account is created, the user is able to access the account and any data associated with the account by entering the identification/contact information in order to identify themselves to the program. Alternatively, the login information is able to be omitted and a user is able to use the program without creating a user profile or logging in.

In some embodiments, upon creation of an account, the login and registration module assigns the account/device 102 a unique identifier (e.g. unique alpha numeric string) that is able to be used to identify the account and/or device 102 of the user that created the account/owns the device 102. Additionally, as described below, this unique identifier is able to be used as the tag for MQTT messages generated by the account/device 102 and/or generated by another account/device 104 where the account/device 102 is the desired destination of the message. The login and registration module is further able to associate the unique identifier within one or more advisor device 104 (and/or the accounts associated therewith) to allow a safe and secure way to communicate between the advisor and client devices/accounts. In some embodiments, the advisor device 104/account is able to invite one or more users (e.g. 4 users) to share this unique client device alpha numeric identifier, so the invited users/accounts/devices 104 are able to communicate with the client device in a closed network. For example, by using the unique identifier as the MQTT tag for any MQTT communications, each of the accounts/devices 102, 104 associated (e.g. subscribed) to the tag/identifier are able to receive and send communications to the other invited users/primary client.

The translation module enables a user to specify (e.g. via the touchscreen or microphone of the graphical user interface) a language and/or dialect to be associated with an account of the program 99 and/or the client device 102. The translation module stores this selected language/dialect preference data and automatically translates incoming audio and/or text messages from their source language to the selected language and/or dialect and then provides the translation of the messages (upon request by a user, for example, via the messages module) to the user of the client device 102. For example, if the message is a text message, the translation module is able to provide translated text on the graphical user interface (e.g. in the speech to text box or elsewhere on the display 202), or if the message is audio, the translation module is able to provide translated audio (in the selected dialect) for output via the speakers 208. Additionally, because the language/dialect preference data is able to be associated with an account, multiple accounts on the same device 102 are able to have different selected languages/dialects, wherein the translation module translates the messages based on which account is currently active/logged into on the device 102 (and adjusts the translation if necessary when the active account switches to another account). Accordingly, the translation module provides the advantage that, for each of the modules and/or functions of the digital assistance program 99, the module is able to ensure all the communications received and/or presented visually and/or auditorily on the device 102 are translated into the selected language and/or dialect.

The calendar module generates the calendar tile 402 that displays one or more of a current time, a current day of the week, and a current date. The calendar module is able to determine the location of the client device 102 (e.g. via the network 108) in order to determine the correct time, day and date values. For example, the calendar module is able to access location, time and date information from the dashboard program 98 of an advisor device 104 (via the network 108), wherein the dashboard program 98 stores a file associated with the unique identifier of the client device 102/account including the location, time and date information. In some embodiments, the user of the dashboard program 98 is able to enter and associate this information with the unique identifier as a part of a client setup process after the unique identifier is associated with the advisor device 104/account (e.g. by the login and registration module). Further, the calendar module is able to include a digital playback button/symbol, wherein when the button is selected by the user, the calendar module outputs audio indicating the time, day and date from the speakers 208. The selection of the digital playback button/symbol is able to be performed by a user touching the touchscreen 202 at a location of the button/symbol and/or upon the microphone 206 receiving audio input indicating a time audio command (e.g. saying the words “Time” or “Date”).

The weather module generates the weather tile 404 that displays one or more of a current temperature, a current weather condition and images or words indicating suggested clothing (e.g. based on the current weather, forecasted weather for the current day, current humidity and/or current temperature). Specifically, the weather module is able to determine the current location, current weather, forecasted weather, current humidity and/or current temperature of the client device 102 (e.g. via the network 108). For example, the weather module is able to access one or more of the above values from the dashboard program 98 of an advisor device 104 (via the network 108), wherein the dashboard program 98 stores a file associated with the unique identifier of the client device 102/account including at least the location of the client device 102. Alternatively, the wether module is able to determine one or more of the above values based on a third party device at a network accessible location (e.g. a weather service website/server where the weather module submits the current location of the device 102 to the website/server in order to retrieve one or more of the above weather values at that location).

In some embodiments, the weather module comprises a generative artificial intelligence (AI) weather agent that inputs the current weather, forecasted weather, current humidity and/or current temperature (or determines said values based on the current location of the device 102 and/or one or more network 108 accessible locations having location-based weather data). In such embodiments, the weather generative artificial intelligence agent is able to use a model (and/or knowledge base) to determine the suggested clothing displayed on the weather tile 404 for the user for that day/time based on the input current weather, forecasted weather, current humidity and/or current temperature. For example, the weather generative artificial intelligence agent is able to suggest clothing that is associated with one or both of the current weather/temperature/humidity (e.g. rain/65 degrees Fahrenheit/96% humidity) and the weather forecast for later in the day (e.g. sunny/70 degrees Fahrenheit/66% humidity). In some embodiments, the weather generative artificial intelligence agent suggests both a first set of clothing based on the current temperature, humidity and/or current weather and a second set of clothing based on the weather, humidity and/or temperature forecast for later in the day. Thus, the weather module provides the advantage of visually and/or auditorily generating clothing suggestions on the digital assistance program 99 based one or both of current and future weather conditions. In some embodiments, the generative AI weather agent is formed by augmenting a general large language model with weather language and/or data.

Like with the calendar module, in some embodiments the user of the dashboard program 98 is able to enter and associate this information with the unique identifier as a part of a client setup process after the unique identifier is associated with the advisor device 104/account (e.g. by the login and registration module). Also similar to the calendar module, the weather module is able to include a digital playback button/symbol, wherein when the button is selected by the user, the weather module outputs audio indicating the current and forecasted weather from the speakers 208. The selection of the digital playback button/symbol is able to be performed by a user touching the touchscreen 202 at a location of the button/symbol and/or upon the microphone 206 receiving audio input indicating a weather audio command (e.g. saying the word “weather”).

The activities module generates an daily activities tile 406 that displays a digital daily activity button for each of the activities scheduled for the current day along with a scheduled time of the activity and a label describing the activity. For example, as shown in FIG. 4, the daily activities tile 406 includes three activities each associated with a label (doctor; lunch; music) a time (7:30; 13:25; 19:45) and a digital button. More or less activities are able to be included each day, wherein the activities are automatically changed/updated by the module upon reaching the corresponding day. In some embodiments, the activities data for each day (e.g. the date, time and label) are stored locally. Alternatively, the activity data for one or more of the activities is able to be received over the network 108 from a third party device or an advisor device 104 (using the add activities module describe below) via the broker 106. In some embodiments, one or more of the activities are able to be set to repeat periodically according to an input schedule/frequency. For example, a lunch activity is able to repeat every day at the same time and/or a music class activity is able to repeat once a week (e.g. every Wednesday at the same time). When any one of the activities has been completed and the associated digital activity button is selected by the user (by touching the screen 202 or via an audio command (identifying the activity as complete), the activities module is able to send a message (via the broker 106) to the advisor program 98 of any advisor devices 104 subscribed to messages from the account/client device 102 that indicates that the one of the activities has been completed.

In some embodiments, the client is able to select the associated button/icon to cause the activities module to issue a text and/or visual reminder of the scheduled activity and/or the starting time of the activity. In some embodiments, any time the client selects the button/icon of an activity, the activities module sends a message to one or more associated advisor devices 104 (e.g. that are subscribed to the unique identifier of the client account/device 102) to let them know the client has reviewed the activity. In some embodiments, the client is able to automatically assign the activity based on the activity name.

The call module generates a call tile 408 that displays a digital call button. The selection of the digital call button is able to be performed by a user touching the touchscreen 202 at a location of the button and/or upon the microphone 206 receiving audio input indicating a call audio command (e.g. saying the word “call” or “help”). When the digital call button is selected, the call module executes a telephone call, video call or text message to one or more advisor devices 104 designated as primary or trusted advisors of the account/client device 102. For example, once the call button has been selected, the call module is able to display (on the display 204) pictures and/or names of the persons of the associated advisor devices 104 and enables the user to select the picture and/or name of the person they would like to call. Once the advisor's picture and/or name is selected, the call connects to that device 104/person with no other action required by the client. If the client should say or yell the word “Help” or other keyword, the call module is able to automatically dial all associated advisor devices 104 and place them on a conference call autocratically so hopefully one of them can get the needed help to the client that is required. In some embodiments, the designated primary and/or trusted advisor devices 104 of the account/client device 102 and the pictures/names thereof are stored on the memory of the client device 102. In some embodiments, when incoming calls are received from an associated advisor device 104, the call module is able to automatically answer the call and start outputting audio of the call so that the client does not need to do anything to answer the call.

The next activities module generates a next activity tile 410 that displays a digital next activities button. The selection of the digital next activity button is able to be performed by a user touching the touchscreen 202 at a location of the button and/or upon the microphone 206 receiving audio input indicating a next activities audio command (e.g. saying the words “next activities”). When the digital next activities button is selected, the next activities module outputs audio indicating a list of the next activities associated with the account/client device 102 from the speakers 208. In some embodiments, the next activities are determined as the activities scheduled for a time within a predefined period after the current time (e.g. 24 hours after the current time). In some embodiments, the activities current displayed within the daily activities tile 406 are excluded from the list of next activities. Alternatively, instead of an audio message, the next activities module is able to output a text message indicating the list of next activities on the display 204 via the graphical user interface (e.g. in the speech to text box 416).

Similarly, the messages module generates a messages tile 412 that displays a digital messages button. The selection of the digital messages button is able to be performed by a user touching the touchscreen 202 at a location of the button and/or upon the microphone 206 receiving audio input indicating a messages audio command (e.g. saying the word “messages”). When the digital messages button is selected, the messages module outputs audio indicating any audio and/or text messages that are associated with the account/client device 102 (that have not yet been listened to or read and/or have been previously heard/read but not deleted) from the speakers 208. In some embodiments, the messages to be output are determined as the messages received at a time within a predefined period before the current time (e.g. 24 hours before the current time). Alternatively, instead of an audio message, the messages module is able to output a text message indicating the messages to be output on the display 204 via the graphical user interface (e.g. in the speech to text box 416).

The digital assistant module generates a digital assistant tile 414 that displays a digital assistant button. The selection of the digital assistant button is able to be performed by a user touching the touchscreen 202 at a location of the button and/or upon the microphone 206 receiving audio input indicating a digital assistant audio command (e.g. saying the word “assistant”). When the digital assistant button is selected, the digital assistant module inputs a question (e.g. text via the text box 416 or audio via the microphone 206) and responds to the question using an artificial intelligence response model. The response is able to be an audio output via the speakers 208 or a text output via the graphical user interface (e.g. text box 416). In other words, in some embodiments the digital assistant module is able to provide an interactive generative artificial intelligence (AI) based Digital Assistant to answer questions via Voice to Text and then convert the Text based response into the native language voice.

For example, the digital assistant module is able to include a general purpose AI agent that answers any question the user has an interest in knowing based on a general purpose model and/or knowledge base. Even if the user language is set in English (via the translation module), the general purpose AI agent is able to understand any language audio input and respond with an answer in the language that has been specified for the account/device 102. Thus, if you have an English speaking caregiver of the user, but the user language is set to Spanish, the caregiver can ask a question in English and the module will still answer the question in Spanish.

In some embodiments, the digital assistant module is able to further comprise a generative Al memory agent that allows the advisor account/device 104 associated with a user account/client device 102 (and/or the client account/device 102 itself) to create a user life story to help those users with cognitive memory issues. In particular, the life story data is able to be stored on the user device 102 and be accessed by the AI memory agent to answer life questions from the user. For example, the life data is able to include name, history, occupations, significant dates, current health conditions, former places of residence, birthplace, ancestry, ethnicity, religion, family relationships, pictures/names of friends/family, address, age and/or other types of user life data. In this way, the user is able to use the digital assistant button to ask questions such as: “what is my name?”, or “when and where was I born?” or Am I married with children?” The memory agent is then able to response to the questions based on a response model and/or the stored life story data (e.g. as a part of a personal knowledge base for the client account/device 102). For example, the digital assistant module is able to output audio stating: “Hello [users' name] and you are [xx] years old born on [month/day/year] . . . ” Also, in some embodiments, when the digital assistant button is selected, a video is played with the digital assistant module outputting the prompt: “What is your question?” and then inputting the user response as they ask their question. In some embodiments, the generative AI memory agent is formed by augmenting a general large language model with the life story data and/or language.

The speech to text module generates a speech to text box 416 that displays text from incoming text, email, video and/or audio messages and/or text from a selected document, book or web page. Alternatively or in addition, the speech to text module is able to automatically output audio reading or playing the incoming text, email, video and/or audio messages and/or text from a selected document, book or web page. In some embodiments, when audio is being received and/or video is being displayed via the display window 418, the speech to text module is able to automatically display text captions of the words being spoken in the audio and/or video within the text box 416.

The display module generates a display window 418 that displays images, video and/or text being received via the network 108 and/or stored on the client device 102. The display module enables a user to control the displayed image/video by a user touching the touchscreen 202 at a location of the display window 418 and/or upon the microphone 206 receiving audio input indicating a display control audio command (e.g. saying the words “big,” “full screen,” “stop,” “play,” etc.). For example, upon receiving a “full screen” command, the display module is able to increase the size of the display window 418 to match the size of the whole display 204, and upon receiving a “minimize” command, the module is able to reduce the size of the display window 418 to a smaller size. Similarly, upon receiving a “play” or “stop” command, the module is able to being playing or stop playing of a video currently displayed in the window 418.

The sensor module is configured to receive data input by the sensors/sensor pad 212 and generate health check-up data to be associated with the account/client device 102 and/or transmitted to one or more of the advisor devices 104 subscribed to the account/client device 102 (and/or the health check-up data from that client/account). For example, the sensors 212 are able to measure attributes of the user when the user presses a finger against the sensor pad 212. In some embodiments, the sensors and/or sensor pad 212 are configured to output a light signal to a user's finger (pressed against the pad 212) and then input reflected and/or transmitted light caused by the user's finger. Based on properties of the reflected and/or transmitted light, the sensors/sensor module is able to determine one or more health values/data about the user. For example, the sensors 212 are able to determine one or more of: user body temperature data; heart rhythm data (e.g. to check for atrial fibrillation); blood-oxygen data (e.g. via a pulse oximeter or SpO2 sensor that measures an amount of oxygen in the blood of the user based on how much light passes through a finger pressed against the sensor pad 212); heart rate data (e.g. pulse rate based on small changes in blood volume within user's arteries caused by user's heartbeat indicated by reflections of light waves emitted into the skin at the sensor pad 212); and blood pressure data (e.g. pulse transit time via electrocardiogram sensor or optical heart rate sensor). This input data is able to be stored and/or used by the sensor module to determine current health values of the user including one or more of body temperature, atrial fibrillation, blood-oxygen levels, heart rate, and/or blood pressure.

In some embodiments, the sensor module is configured to output a health check instruction video on the display 204 upon receiving a request for help from the user, wherein the instruction video describes what the user needs to do for the device 102 to input/measure the user's health data. In some embodiments, the sensor module is configured to output the health data to the user on the display 204 upon completion of the health check and/or transmit the heath data to one or more advisor devices 104/accounts associated with the unique identifier of the user account/client device 102.

The communication module enables the client device 102 (or an advisor device 104) to communicate data and/or messages to and receive data/messages from the broker device 106. Specifically, the communication module is able to implement a modified message queue telemetry transport (MQTT) communication protocol that it is able to use when one of the other modules or components of the client device 102 want to send data to or receive data from one of the advisor devices 104 (and/or another device).

This modified MQTT protocol is able to define two types of network entities: a message broker device 106 and a number of client/advisor devices 102, 104. The MQTT broker device 106 receives all messages from the clients/advisor devices 102, 104 and then routes the messages to the appropriate destination devices 102, 104. Information/messages are able to be organized in a hierarchy of topics or identifiers added as a topic tag to each of the messages. When a publisher (e.g. client/advisor 102, 104) has a new item of data to distribute, the communication module of the publisher is able to construct and send an MQTT message (with the item of data formatted as the payload of the message and a topic/identifier as the tag of the message) to the broker 106. The broker 106 then distributes/publishes the payload data to any clients/advisor devices 102, 104 that have subscribed to the topic identified by the topic tag. Unlike other communication protocols, this modified protocol has the benefit of enabling bi-directional communication as each client/advisor device 102, 104 is able to both produce and receive data by both publishing and subscribing.

The topic/topic tag generated by the communication module is able to be the unique identifier of a client account/device 102 or an advisor account/device 104. Alternatively, the topic tag is able to be another value (e.g. a topic of the message). In some embodiments, the unique identifier of a client account/device 102 is able to serve as the topic tag for all the communications related to that client account/device 102 (e.g. messages sent from the client device 102 and/or messages sent from advisor devices 104 related to the client account/device 102). As a result, other devices 102, 104 that did not publish the message, but are interested in receiving data associated with that client account/device or advisor account/device are able to subscribe to that unique identifier/topic tag. For example, an advisor device 104 (e.g. family or care giver account/provider) is able to subscribe to the unique identifier tag/topic of a client device 102 (e.g. patient/client account) in order to receive and monitor messages from and/or relating to that client account/device 102. The publisher does not need to have any data on the number or locations of subscribers, and subscribers, in turn, do not have to be configured with any data about the publishers. Multiple clients and/or advisor devices 102, 104 are able to subscribe to a topic/unique identifier of a single client 102 or advisor 104 (many to one capability), and a single client or advisor device 102, 104 is able to subscribe to topics/unique identifiers of multiple clients 102 or advisors 104 (one to many).

In the case of caregiving, this can be in the form of a set of trusted advisor devices 104 (e.g. friends, family, caregivers) all being subscribed to the unique identifier/tag of a patient/client account/device 102 such that they are all able to communicate with and/or about the client using that unique identifier as the message tag. Similarly, a single trusted advisor device 104 (e.g. healthcare professional) is able to subscribe to the unique identifiers of multiple client/patient devices 102 such that they are able to provide care to each of those clients 102 by selecting the appropriate unique identifiers for their communications/subscriptions. Further, in some embodiments an identifier/tag is able to be associated with a plurality of client accounts/devices 102 such that a single trusted advisor device 104 is able to publish a message that will be received by all of the different clients 102 (which are all subscribed to that identifier/tag). Moreover, the network of client and advisor devices 102, 104 is able to include combinations of the above configurations. For example, a single advisor 104 associated with multiple clients 102, wherein one or more of the clients 102 are associated with their own personal set of other advisor devices 104 (e.g. family/friends). This modified protocol provides the benefit of a force multiplying effect with regard to communications.

In order to construct one or more of the MQTT messages, the communication module needs to input data to be transmitted and convert the input data into a format that is able to be received by the modified MQTT protocol. In particular, in some embodiments the modified MQTT protocol requires the payload of the MQTT messages to be in a text or character based format (e.g. ASCII format). Thus, in order to facilitate the transmission of non-text and/or character based data (e.g. media data), the communication module needs to input the data (received in various formats depending on the type of data and the source of the data) and then standardize the input data into a standard text based format that is compatible with the MQTT message payload requirements. For example, the communication module is able to perform the standardization by inputting the non-text based formatted data (e.g. media data and/or non-character/text data), encoding the non-text based formatted data using an encoding metric (e.g. base64) into encoded data, and then converting the encoded data to a text/character based format (e.g. ASCII). The module is then able to insert the converted data (now in a standardized format) as payload within an MQTT message for transmission over the network 108 to one or more other devices 102, 104 (via the broker 106).

The non-text and/or character based input data is able to be audio (e.g. speech, music), images, sensor data (e.g. health data obtained from the patient/client via the sensors 212), video, other non-text based format data or a combination thereof. In particular, the data is able to have many varied and/or different formats (e.g. .mp3, .wav, .mp4, json, .png, .jpg) and/or be unformatted. Thus, the communication module provides the advantage of being able to receive media data having multiple different formats and standardizing the data into a single character-based format such that it is able to be transmitted via the MQTT message protocol. In some embodiments, some or all of the data is input via the client and/or advisor device 102, 104 such that the data is able to be input via a file stored on the memory of the device 102, 104, the microphone 206, the camera 210 and/or the sensors/pad 212 and/or other input interfaces of the device 102, 104.

Subsequently, after the MQTT message payload is delivered to the subscribed devices (via the broker 106), the receiving communication module of each of the receiving devices 102, 104 is able to perform the reverse process on the received payload data to revert the data to its original format. Specifically, the communication module is able to convert received payload in its text/character format back into its encoded non-text based format, and then decode the data from the encoded non-text format back into the un-encoded original/input data (e.g. with its correction file extension). As a result, the receiving device 102, 104 is then able to utilize the un-encoded original/input data to play audio files, graphically display the sensor data, and/or view the video or picture files of the input data on the receiving device 102, 104.

In some embodiments, a translation unit of the communication module is able to receive the input data (which as received is sometimes segmented into chunks of a first size (e.g. 8-bit chunks)) and parse the input data into a number of equal size chunks based on the size required by a desired binary encoding algorithm (e.g. Base32, Base64, Base 128, Base 256, etc.). In some embodiments, the binary encoding algorithm is Base64. In particular, Base 64 offers a sufficient number of characters to support a wide range of applications but still is limited to characters that are generally recognized by most systems. Thus, if the desired binary encoding algorithm is Base64, the translation unit is able to parse the input data stream into 6-bit chunks or segments. Alternatively, the binary encoding algorithm is able to be Base32, Base 256 or other binary encodings that provide an ASCII equivalent representation and/or the parsing is able to be performed based on the requirements of the chosen encoding algorithm (e.g. 5-bit chunks for Base32).

In some embodiments, an encoding unit of the communication module receives the chunks of data from the translation unit and converts each of the chunks into a character of a selected character-based format (e.g. ASCII format). For example, if ASCII is the desired character-based format, for each of the chunks of input data, the encoding unit is able to determine an ASCII character that corresponds to the bits of the chunk. Specifically, the encoding unit is able to utilize a conversion table that maps each possible combination of bits in a chunk to a unique ASCII character. In some embodiments, the encoding unit is able to first convert the chunks to a decimal value, and then determine an ASCII character that maps to that decimal value in the conversion table. Alternatively, the encoding unit is able to directly determine which ASCII character maps to each chunk of bits from the conversion table. In some embodiments, the size of the conversion table (which is able to be stored on the device 102, 104) is able to be based on the desired/selected binary encoding algorithm. Thus, if Base64 is selected, the table is able to have 64 unique (e.g. ASCII) characters mapped to the different possible bit permutations of the 6-bit chunks. Alternatively, if Base32 or another size encoding algorithm is used, the conversion table is able to be adjusted such that there is a unique (e.g. ASCII) character mapped to each possible bit combination for the chunk size (required for that algorithm).

In some embodiments, a sending unit of the communication module adds the data (now in the character/text based format) as payload in an MQTT message, adds a tag to the message (e.g. a unique identifier or topic) and transmits the message to the broker device 106. As a result, other devices (e.g. advisor devices 104) that are subscribed to that unique identification/tag are able to receive the MQTT message from the broker device 106.

Again, upon receiving the MQTT message, the communication module of each of the receiving devices 102, 104 is able to reverse the encoding/converting process described above to obtain and output the original media data for users of the receiving device 102, 104. In particular, the sending unit is able to input the MQTT message and parse the payload data (in the character/text format), the encoding unit is able to convert the payload data from the character format (e.g. ASCII format) back to binary chunks based on the conversion table and/or the encoding algorithm, and the translation unit is able to convert the chunks back to the original media data. The original data then is able to be output by the receiving device 102, 104, for example, via the speakers 208, the display 204, the display window 418, the speech to text box 416 and/or a combination thereof that is suitable for the type of media data (e.g. image, video, audio, etc.).

In some embodiments, if the encoding algorithm is Base64, the conversion table is able to be the Base64 alphabet defined in RFC 4648 § 4. In some embodiments, the encoding unit is able to convert the chunks received from the translation unit to the ASCII hexadecimal values that map to the combination of bits of each chunk (and/or the decimal value thereof) for storage as payload of one or more MQTT messages. For example, a chunk bit combination or decimal value that corresponds to the character “A” in the conversion table, is able to be converted into a hex value (e.g. 0x41) within the table that corresponds to the character “A” (and the chunk bit combination or equivalent decimal value). In some embodiments, the program 99, 98 of the receiving device 102, 104 is able to generate another message based on the received original data. For example, an alarm is able to be issued based on the receipt of the original data.

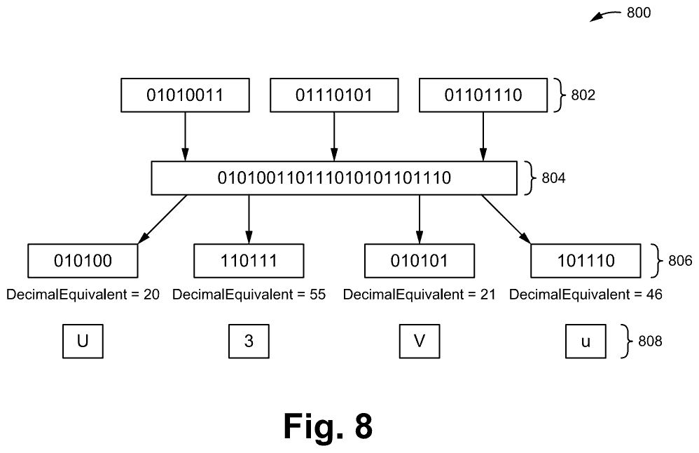

FIG. 8 illustrates an exemplary communication module data conversion process 800 according to some embodiments. As shown in FIG. 8, a stream of non-text/character based data 802 is input by the communication module. In some embodiments, the data 802 is received in segments having an original size (e.g. 8 bits). Alternatively, the data 802 is able to be received in other size segments and/or unsegmented. The data 802 is able to be grouped or concatenated by the communication module into a raw data segment 804 having a predetermined size (e.g. 24 bits). Specifically, the predetermined size of the raw data segment 804 is able to be equal to a multiple of the chunk size required by the desired encoding algorithm. Thus, if base64 is the desired encoding algorithm, the bit size of the raw data segment 804 is able to be a multiple of 6. Alternatively, other segment sizes are able to be used and/or the grouping is able to be omitted.

The communication module parses the raw data segment 804 into one or more equal size chunks 806. As described above, the size of the chunks 806 is able to be based on the desired encoding algorithm. The communication module then converts each of the chunks 806 into a corresponding character 808 of a selected character-based protocol (e.g. ASCII) based on a stored conversion table (that corresponds to the selected character-based protocol and/or encoding algorithm). In particular, the table is able to map binary and/or decimal numbers to a set of characters such that each possible permutation of bits of the chunks has a corresponding unique character. Thus, as shown in FIG. 8, a first chunk 806 corresponds to the bits 010100 (i.e. decimal 20), wherein in the conversion table (e.g. ASCII base64 table) the bits 010100 (and decimal 20) correspond to the character “U” 808. Similarly, the three other chunks 806 correspond to the characters “3,” “V,” and “u,” respectively.

As a result, the communication module provides the benefit of enabling a large quantity of clients/advisor devices to be in communication via various media data of different file formats with minimal overhead, faster response times and better scalability. This solves the technical problem of previous HTTP based systems that can only respond to data requests (uni-directional data flow), require a lot of overhead, increasing the bandwidth requirements, require the user to open and close a connection each time a data packet is sent, and have bandwidth limited scalability. Additionally, it should be noted that although the communication module is described here with respect to the digital assistance program 99 on a client device 102, the communication module is able to be a part of and operate in the same manner in the dashboard program 98 on advisor devices 104.

As described above, the broker (e.g. broker device 106) is able to act as a central hub that manages communication between devices 102, 104 in a publish/subscribe messaging system. It acts as an intermediary, receiving messages from publishers and distributing them to subscribers based on their topic subscriptions. The broker's responsibilities are able to include one or more of: Receiving and filtering messages, Identifying devices 102, 104 subscribed to each message, implementing quality of service (QOS) on a per tag basis, establishing persistence connections to automatically keep a connection pathway open, sending messages to subscribers, handling large numbers of concurrent connections, and ensuring reliable message delivery. In some embodiments, the broker is able to support millions of concurrently connected devices 102, 104 which provides the ability establish secure services among small groups of clients or broadcast configurations that allow one publishing device 102, 104 to reach an unlimited number of subscribers.

### The Dashboard Program

The dashboard program 98 is able to comprise a login and registration module, a translation module, an add activities module, a call module, an audio messages module, a media messages module, a text to speech module, an entertainment module, a health check module, a client view module, a translation module and a communication module. Alternatively, one or more of the modules are able to be omitted and/or combined into a single module. The functions of one or more of the modules are accessible to users via a dashboard graphical user interface displayed on a display of the advisor device 104. For example, the modules are able to receive input via the graphical user interface and perform functions described herein based on the input. Additionally, it should be noted that it is contemplated that although described separately, some or all of the modules of the dashboard program 98 described herein are able to be included in the digital assistance program 99, and/or some or all of the modules of the digital assistance program 99 described herein are able to be included in the dashboard program 98.

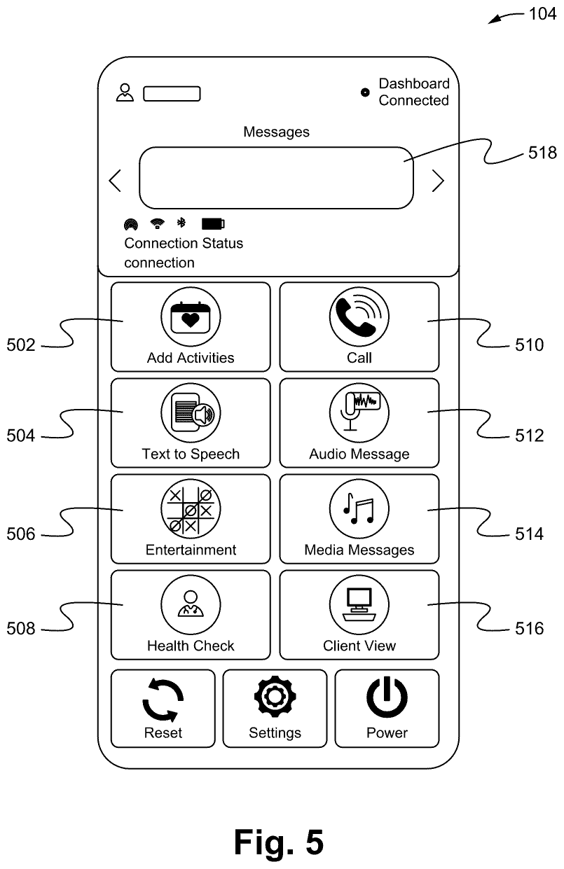

FIG. 5 illustrates an exemplary screenshot of the graphical user interface as displayed on an advisor device 104 according to some embodiments. As shown in FIG. 5, the graphical user interface of the dashboard program 98 is able to comprise an add activities tile 502, a text to speech tile 504, an entertainment tile 506, a health check tile 508, a call tile 510, an audio messages tile, a media messages tile 514, a client view tile 516 and a text box 518. Alternatively, one or more of the components of the graphical user interface shown in FIG. 5 are able to be omitted. Additionally, the relative size and/or position of each of the components is able to be dynamically adjusted.

The login and registration module and communication module of the dashboard program 98 is able to be substantially the same as the login and registration module and communication module of the digital assistance program 99 described above and thus not repeated here for the sake of brevity.

The translation module enables a user to specify (e.g. via the touchscreen or microphone of the graphical user interface) a language and/or dialect to be associated with an account of the program 98 and/or the advisor device 104. The translation module stores this selected language/dialect preference data and automatically translates incoming audio and/or text messages from their source language to the selected language and/or dialect and then provides the translation of the messages (upon request by a user, for example, via the messages module) to the user of the advisor device 104. For example, if the message is a text message, the translation module is able to provide translated text on the graphical user interface (e.g. in the text box 518 or elsewhere), or if the message is audio, the translation module is able to provide translated audio (in the selected dialect) for output via speakers of the device 104. Additionally, because the language/dialect preference data is able to be associated with an account, multiple accounts on the same device 104 are able to have different selected languages/dialects, wherein the translation module translates the messages based on which account is currently active/logged into on the device 104 (and adjusts the translation if necessary when the active account switches to another account).

The add activities module generates an activities tile 502 that displays an add activities button. Upon selection of the add activities button, the add activities module enables the user to input one or more activities to add to a client account, wherein each activity includes an input label describing an activity, a time and date of the activity, an identifier of a client account to add the activity to, and/or a specification of whether the activity repeats/a frequency of the repetition. The add activities module automatically passes this new activity data to the communications module which then includes the data as payload data in a MQTT message having a tag identifying the client account such that the client device 102 subscribed to that account/tag receives the new activity data and (via the activities module) adds the new activities to the daily activities tile 406 at the indicated time and date of the activity. In some embodiments, the add activities module enables the user to add media files (e.g. pictures, video, music, audio books, health requests, text passages from books/poems/etc.) to one or more of the activities (wherein the added media data of the media files is transmitted to the client device 102 via the communication module as described herein). Upon selection or performance of these activities having the added media on the client device 102 (upon reaching the scheduled time), the digital assistant program 99 of that device 102 is able to play the added media on the device 102 (e.g. via the display 204, text box 416, display window 418 and/or speakers 208) to facilitate the performance of the activity. For example, a music activity is able to include the playing of added music media added to the activity by the advisor account/device 104.

The text to speech module generates a text to speech tile 504 including a text to speech button. When text is input in the text box 518 (e.g via a keyboard coupled with the device 104 and/or a virtual keyboard of the GUI of the dashboard program 98) and the text to speech button is selected, the text to speech module transmits the input text to the associated client account/device 102. In some embodiments, if the advisor device 104/account is associated with multiple client accounts/devices 102, the text to speech module presents the associated client accounts/devices 102 and transmits the input text after selection of the desired client account/client device 102. As described above with respect to the speech to text module of the digital assistance program 99, upon receipt of the text message, the speech to text module is able to display a note in the text box 416 that there is an incoming message and/or simultaneously play an audio and/or video announcement that there is an incoming message. In some embodiments, after playing the message there is a pause (e.g. 10 seconds) to give the client time to come to the device 102 and then the text message will be displayed and played in the text box 416 and/or output via the speakers 208.

The call module generates a call tile 510 that displays a digital call button. When the digital call button is selected, the call module executes a telephone call, video call and enables the capabilities to share a document and stream a video to one or more client devices 102 designated (e.g. stored in local memory) as clients, patients or relatives of the account/advisor device 104. In some embodiments, the designated client devices 102 associated with the account/advisor device 102 are stored on the memory of the advisor device 104. As described above, in some embodiments the call module of the digital assistance program 99 is configured to automatically answer the calls from the associated advisor accounts/devices 104. In some embodiments, the call module of the client device 102 automatically displays an image of the user/caller of the advisor device 104 on the screen 204 of the client device 102, and the call module of the advisor device 104 automatically displays an image of the user of the client device 102 via the GUI of the dashboard program 98.

The entertainment module generates entertainment tile 506 that displays an entertainment button. When the entertainment button is selected, the module enables a user to select and schedule a time to play a game with one or more of the associated client accounts/devices 102. For example, the module is able to enable selection of a game and/or time that is able to be added as a scheduled activity for the client account/device 102.

The audio messages module generates an audio message tile 512 including an audio message button. Upon selection of the audio message button, the audio messages module enables selection of a “natural voice” option or a “translated message” option and upon selection of one of the options begins recording audio via a microphone of the device 104 (e.g. for transmission to a client device 102 via the communications module) and transmits the completed recording to a selected client account/device 102 (e.g. identified by the unique identifier). When the “natural voice” option is selected, the digital assistance program 99 of the receiving client device 102 is able to indicate (e.g. in the text box 416) that an incoming message has been received and/or play audio and/or video indicating that there is a new message. In some embodiments, after the audio and/or video ends, the device 102 pauses for a period (e.g. 10 seconds) to allow the client to come to the device 102 and then it plays the voice message in the advisors natural voice (i.e. without translation). When the “translation” option is selected, the user of the advisor device 104 is able to select a desired translation language in addition to recording the message (in their native language). Upon receiving an audio message with the “translation” option selected, the digital assistance program 99 of the receiving client device 102 is able to operate in the same manner as with the “natural voice” option except with the audio translated into the desired translation language.

In some embodiments, upon input of a command (e.g. via the graphical user interface) indicating completion of the audio, the audio messages module prompts the user to specify a tag/account unique identifier as the desired destination for the message. As described above, the communication module is able to convert this non-ASCII format media data to an ASCII format such that it is able to form the payload of an MQTT message.

The media messages module generates a media message tile 514 including a media message button. Upon selection of the media message button, the media messages module enables a user to input one or more media files (e.g. pictures, videos, music, books, audio books, etc.) that the module then sends to a desired client account/device 102 (e.g. via the communication module). Upon receipt of the media files, he digital assistance program 99 of the receiving client device 102 is able to indicate (e.g. in the text box 416) that an incoming message has been received and/or begin playing the media audio and/or video indicating that there is a new message. In some embodiments, after the audio and/or video ends, the device 102 pauses for a period (e.g. 10 seconds) to allow the client to come to the device 102 and then it plays the media files on the device 102. In some embodiments, upon input of a command (e.g. via the graphical user interface) indicating completion of submission/selection of the media files, the media messages module prompts the user to specify a tag/account unique identifier as the desired destination for the message. Again as described above, the communication module is able to convert this non-ASCII format media data to an ASCII format such that it is able to form the payload of an MQTT message.

The client view module generates a client view tile 516 including a client view button. Upon selection of the client view button, the client view module displays a virtual view (e.g. screen shot) of the graphical user interface of the digital assistance program 99 for the associated client device/account. In some embodiments, the client view module is able to determine which client account/device 102 to retrieve a virtual view from based on one or more stored unique identifiers associated with the advisor account of the advisor device 104 that are stored in a memory of the advisor device 104. As a result, the user of the client view module is able to “check in” on wellness of the clients and/or the status of the digital assistance programs 99 of one or more associated accounts/client devices 102.

The health check module generates a health check tile 508 including a health check button. Upon selection of the health check button, the health check module enables the user to decide which of a set of advisor accounts/devices 104 associated with the desired target client 102 (e.g. in their “trusted” group) will be sent a message (e.g. email) with the results of the Health check. After this data is input by the user, the health check module enables the user to initiate an instant heath check or schedule the Health check on as a future activity for the client account/device 102. If the schedule option is selected, the module enables the user to input a date and time to schedule the health check up (which is then able to be transmitted to the client device 102 and added to the list of activities for the client on the client device).

If the instant health check option is selected (or upon reaching the scheduled time and date), the health check module sends the health check request message to the client's device 102, which shows an incoming message in the text box 416 and/or plays a Health Check video which indicates to the Client how to perform the health check to successfully record the Health data (as described above in relation to the sensor module 212). Once the health data from the health check is received by the advisor device 104, the health check module is able to display the health data on the GUI of the advisor device 104 (similar to the display on the window 418 of the client's device 102). Additionally, if any were selected upon the creation of the health check up request, the health check module is able to automatically send (e.g. email) the health information to the one or more advisor accounts/devices 104 selected from the set of advisor accounts/devices 104 associated with the desired target client 102 (e.g. in their “trusted” group).

In some embodiments, for the scheduled health check option the local activities module of the client/account is able to automatically add the new health check activity to the list of activities on the display 204 (or to be displayed in the future) upon receipt of the message. In some embodiments, the requested health data is able to include one or more of the health measurements able to be input by the sensors/pad 212 as described above. Alternatively or in addition, the health data is able to be other types of health data that is measured by non-client devices 102 that are coupled with the client device 102 (e.g. bluetooth connected blood pressure and/or blood glucose sensors).

In some embodiments, the dashboard program 98 is able to further comprise a caregiver visit notes module and button, wherein upon selection, the notes module inputs and records audio, text, images and/or video notes about a client/account and then automatically generates a message for the broker 106 including those recorded notes (e.g. audio, text, images and/or video) including a tag that uniquely identifies the client/account such that all other accounts subscribed to that client/account is broadcast the notes.

### Methods of Operation

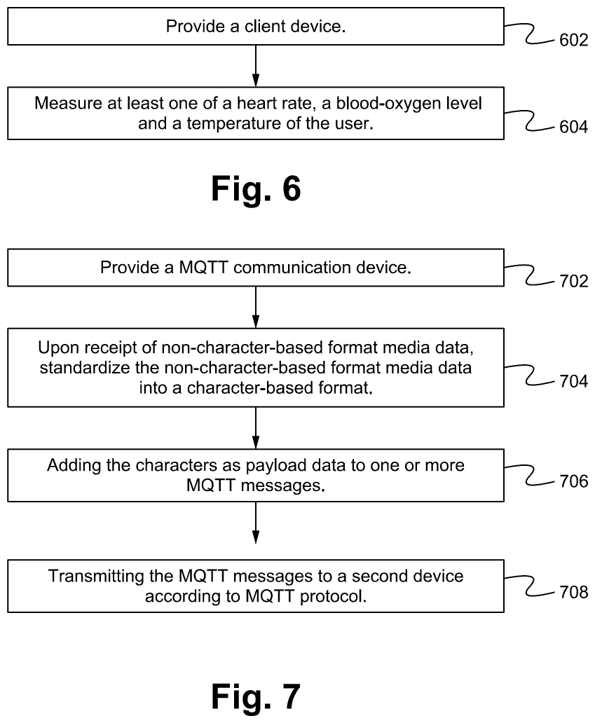

FIG. 6 illustrates a method of providing care to one or more users with client device 102 including a digital assistance program 99 according to some embodiments. As shown in FIG. 6, a client device 102 is provided at the step 602. The client device 102 is able to include the housing 202, the touch screen 204 coupled with the housing 202 for displaying images and receiving touch commands, cameras 210 coupled with the housing 202, microphones 206 coupled with the housing 202, speakers 208 coupled with the housing 202, a finger touch pad and health sensors 212 coupled with the housing 202 for receiving contact from a finger of a user, and a non-transitory computer-readable memory storing the digital assistance program/application 99 having a graphical user interface that is displayed on the touch screen 204. At least one of a heart rate, a blood-oxygen level and a temperature of the user is measured by the sensors/pad 212 at the step 604. The at least one of heart rate, blood-oxygen level and temperature of the user are able to be determined based on measurements received by the sensors via the finger touch pad while being contacted by the finger of the user.

In some embodiments, the method further comprises, upon selection of one of the digital activity buttons, publishing a message (via the broker 106) with the communication module of the digital assistance application 99 to one or more trusted advisor devices 104 that are subscribed to the client device 102, the message identifying the activity as being completed. In some embodiments, the method further comprises, upon receiving a fullscreen command, changing the image/video window from a first size that is smaller than the touch screen 204 to a larger size that is the same size as the touch screen 204 with the display module of the application 98 thereby increasing the size of images or video displayed by the image/video window 418. In some embodiments, the method further comprises, generating images and/or text on the weather tile 404 of the touch screen 204 with the application 99 indicating current suggested clothing and future suggested clothing based on at least one of a current temperature, a current weather, a current humidity and a forecast weather for a location of the assistance device.

In some embodiments, the method further comprises, upon receiving a call command via the call button of the call tile 408, initiating a telephone call with the call module to at least one of the trusted advisor devices 104. In some embodiments, the method further comprises, upon receiving selection of the digital assistant button of the tile 414 and a personal query about the user, outputting an answer to the personal query based on the personal data of the user stored in the personal database with the digital assistant AI memory agent. In some embodiments, the method further comprises, upon receiving a selection of the digital next activities button of the tile 410, displaying a list of upcoming activities associated with the user on the touch screen 204. In some embodiments, the method further comprises, with the communication module and upon receipt of media data having a non-character based format, performing a binary encoding on the media data to convert the media data into one or more corresponding characters of a character based format, adding the corresponding characters as payload data to one or more MQTT messages and transmitting the MQTT messages to a trusted advisor device according to a MQTT protocol. In some embodiments, the method further comprises, with the communication module and upon receipt of an MQTT message from the trusted advisor device, parsing the payload data from the MQTT message, converting characters of the payload data to corresponding the media data having the non-character based format according to the binary encoding, and outputting the media data on at least one of the touchscreen and the speakers with the application.

FIG. 7 illustrates a method for providing a modified MQTT communication program to one or more users according to some embodiments. As shown in FIG. 7, a MQTT communication device 102, 104 is provided at the step 702. The MQTT communication device 102, 104 including an output interface for outputting first media data from the device 102, 104, an input interface for inputting non-character-based format media data and a non-transitory computer-readable memory storing a communication application 99, 98. Upon receipt of non-character-based format media data, the communication application standardizes the non-character-based format media data into a character-based format by converting each chunk of the non-character-based format media data to a character of the character-based format at the step 704. In some embodiments, the method further comprises converting the characters to one or more corresponding hex characters according to the character based format (e.g. ASCII). The communication application adds the characters as payload data to one or more MQTT messages at the step 706. In some embodiments, the communication module further adds a tag to each of the MQTT messages, wherein the tag identifies a destination of the messages (e.g. a destination device, a destination account). Alternatively, the tag is able to identify a subject or topic to which the message relates. The communication application transmits the MQTT messages to a second device 102, 104 according to MQTT protocol at the step 708.

In some embodiments, the method further comprises upon receipt of another MQTT message from the second device 102, 104, converting each of the characters of the payload data of the another MQTT message to a corresponding chunk of non-character-based format media data with the communication application and outputting the corresponding chunks of the non-character-based format media data via the device 102, 104 with the communication application. In some embodiments, the non-character-based format media data comprises at least one of images, video and audio. In some embodiments, the character-based format is ASCII and the converting of each chunk of the non-character-based format media data to the character of the character-based format comprises performing a Base64 encoding the non-character-based format media data.

### Advantages

The system 100, method and device, provides numerous benefits. Specifically, the system helps trusted advisors (e.g. professional assistants, family members, friends, and/or caregivers that are responsible for the client) care for their clients partly remotely, through providing a specialized client digital assistance device 102 that streamlines administration tasks to communicate the well-being, observations, and needs of their clients with family members and medical support teams. In particular, the system improves the problem of inadequate support provided by single-touch fixed in-home visits by, for example, enabling users (e.g. elderly) via the specialized client digital assistance device 102 to submit physical wellness checks using the device 102 built-in wellness sensors (for taking health readings), to submit activity statuses (e.g. complete), to receive reminders of important data (e.g. activities, weather, date, time), and to submit audio and/or video using the device 102 hardware for communicating with trusted advisors 104 (including a digital assistant). Similarly, the caregiving advisor application enables the advisors to quickly check the status of many users, respond to issues and request wellness data and/or other activities from the users (based on the received data from the caregiving device of each of the users). Further, the weather module provides the benefit of suggesting clothing to users for not just current weather/environmental conditions in their location, but also based on projected future conditions (wherein the suggestion is able to include two different types of clothing when the current and predicted weather merit different clothing). Also, the digital assistant module provides the benefit of not only answering general questions, but also storing and answering personal questions related to a history of the client account/device 102. Moreover, the translation module provides the benefit of enabling different languages and/or dialects to be used for creating messages by devices 102, 104 on the system 100, wherein the module ensures that each client/advisor device 102, 104 is able to hear/read the messages in their selected/preferred language and/or dialect (e.g. including having an accent that corresponds to the selected language/dialect).

The system also solves the problem of inadequate assistance GUIs with small icons, text and/or quiet audio that is unable to be effectively used by elderly users with poor eyesight hearing and/or dexterity. Specifically, the assistance GUI described herein solves this problem by including large buttons that respond to both physical touch and audio commands. Further, the GUI includes re-scalable images and/or text boxes that are able to become fullscreen or otherwise increase in size based on selection of a digital button and/or an audio command thereby enabling user to easily read transcriptions of audio messages (and/or other data) as well as easily view received pictures and/or video.

Moreover, the system solves the problem of HTTP based systems that are designed to handle media of various formats and so can only respond to data requests (uni-directional data flow), require a lot of overhead, increasing the bandwidth requirements, require the user to open and close a connection each time a data packet is sent, and have bandwidth limited scalability. In particular, the system is able to encode data received/input in non-standardized formats (e.g. video, audio, etc.), encode the data, and convert the data to a standardized format (e.g. ASCII format) such that it is able to be transmitted as payload using an MQTT protocol thereby gaining the benefits of little overhead, faster response times, automatic/subscription based data transfer and scalability.

Additionally, the system provides the advantage of: utilizing a lightweight and energy efficient protocol stack; allowing the assignment of unique data tags for all message types; providing the ability to send asynchronous data and bidirectional communication; data changes being sent only when a change in data takes place thereby saving network bandwidth; allowing all messages to be supported with SSL/TLS security; enabling different Quality of Service (QOS) assignment for each data tag created; enabling force multiplication due to use of the publish/subscribe communication protocol; unique clients able to be associated with both a single and group of trusted advisors; caregiving facility is able to handle multiple unique clients cared for by a single trusted advisor; senior living center is able to utilize a single trusted advisor to care for multiple clients; and protocol is event driven such that data is shared only when there is a change in the data. Thus, as a result of this innovative media file translation approach that embeds media files into an MQTT text message payload, the system 100 provides users the opportunity to force multiply their caregiving services of elderly clients. It also provides a pathway for Clients to have peace-of-mind in knowing that they are communicating across a secure and closed data network between the client devices 102 and their group of advisor devices 104 (e.g. caregivers, family, and friends). Thus, the caregiving system, method and device, provides numerous benefits.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiments chosen for illustration without departing from the spirit and scope of the invention as defined by the claims. For example, although functions of the programs 99, 98 are generally described with respect to a single client account/device 102 and a single set of associated advisor accounts/devices 104 (such that the unique identifier of the single client account/device 102 is able to be automatically known for all functions), multiple client accounts/devices 102 (and their unique identifiers) are able to be associated with one or more advisor accounts 104. In such embodiments, one or more of the modules of the dashboard program 98 are able to present a list of the associated unique identifiers before any functions that would target one or more of the devices 102 such that the advisor account/device 104 is able to select which of the devices 102 (i.e. unique identifiers) are the desired targets for the function. Thus, after this initial selection, the operation of the modules is able to be the same as described in the case with a single client account/device 102 (where no selection was necessary). Also, like all the modules, the translation unit. encoding unit and sending unit of the communication module are able to be implemented in hardware, software or a combination thereof.

What is claimed is:

1. An assistance device for providing care to one or more users, the assistance device comprising:
a protective housing;
a touch screen coupled with the housing for displaying images and receiving touch commands;
one or more cameras coupled with the housing;
one or more microphones coupled with the housing;
one or more speakers coupled with the housing;
a finger touch pad coupled with the housing for receiving contact from a finger of a user;
one or more sensors operably coupled with the finger touch pad and configured to measure at least one of a heart rate, a blood-oxygen level and a temperature of the user based on measurements received by the sensors via the finger touch pad while being contacted by the finger of the user; and
a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device.

2. The device of claim 1, wherein upon selection of one of the digital activity buttons, the application publishes a message that is received by one or more trusted advisor devices that are subscribed to the assistance device, the message identifying the activity as being completed.

3. The device of claim 1, wherein upon receiving a fullscreen command, the application is configured to change the image/video window from a first size that is smaller than the touch screen to a larger size that is the same size as the touch screen thereby increasing the size of images or video displayed by the image/video window.

4. The device of claim 1, wherein the graphical user interface includes a weather tile and the assistance application includes a generative artificial intelligence weather agent that generates images and/or text on the touch screen indicating current suggested clothing and future suggested clothing based on at least one of a current temperature, a current weather, a current humidity and a forecast weather for a location of the assistance device.

5. The device of claim 1, wherein the graphical user interface further comprises a digital call button that is associated with one or more trusted advisor devices, and upon receiving a call command, the application is configured to initiate a telephone call to at least one of the trusted advisor devices.

6. The device of claim 1, wherein the graphical user interface further comprises a digital assistant button associated with a database of personal data about a life of the user, and upon receiving a personal query about the user, a generative artificial intelligence memory agent of the application outputs an answer to the personal query based on the personal data of the user.

7. The device of claim 1, wherein the graphical user interface further comprises a digital next activities button, and upon receiving a selection of the digital next activities button, the application is configured to display a list of upcoming activities associated with the user.

8. The device of claim 1, wherein the application comprises a communication module, and upon receipt of media data having a non-character based format, the communication module performs a binary encoding on the media data to convert the media data into one or more corresponding characters of a character based format, adds the corresponding characters as payload data to one or more MQTT messages and transmits the MQTT messages to a trusted advisor device according to a MQTT protocol.

9. The device of claim 8, wherein upon receipt of an MQTT message from the trusted advisor device, the communication module parses the payload data from the MQTT message, and converts characters of the payload data to corresponding the media data having the non-character based format according to the binary encoding, and further wherein the application outputs the media data on at least one of the touchscreen and the speakers.

10. The device of claim 8, wherein the media data comprises at least one of images, video and audio.

11. The device of claim 8, wherein the binary encoding is Base64 encoding.

12. An assistance system for providing care to one or more users, the assistance system comprising:
an assistance device including:
a protective housing;
a touch screen coupled with the housing for displaying images and receiving touch commands;
one or more cameras coupled with the housing;
one or more microphones coupled with the housing;
one or more speakers coupled with the housing;
a finger touch pad coupled with the housing for receiving contact from a finger of a user;
one or more sensors operably coupled with the finger touch pad and configured to measure at least one of a heart rate, a blood-oxygen level and a temperature of the user based on measurements received by the sensors via the finger touch pad while being contacted by the finger of the user; and
a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device; and
one or more trusted advisor devices including a trusted advisor application for communicating with the assistance device.

13. The system of claim 12, wherein upon selection of one of the digital activity buttons, the assistance application publishes a message that is received by one or more trusted advisor devices that are subscribed to the assistance device, the message identifying the activity as being completed.

14. The system of claim 12, wherein upon receiving a fullscreen command, the assistance application is configured to change the image/video window from a first size that is smaller than the touch screen to a larger size that is the same size as the touch screen thereby increasing the size of images or video displayed by the image/video window.

15. The system of claim 12, wherein the graphical user interface includes a weather tile and the assistance application includes a generative artificial intelligence weather agent that generates images and/or text on the touch screen indicating current suggested clothing and future suggested clothing based on at least one of a current temperature, a current weather, a current humidity and a forecast weather for a location of the assistance device.

16. The system of claim 12, wherein the graphical user interface further comprises a digital call button that is associated with one or more trusted advisor devices, and upon receiving a call command, the application is configured to initiate a telephone call to at least one of the trusted advisor devices.

17. The system of claim 12, wherein the graphical user interface further comprises a digital assistant button associated with a database of personal data about a life of the user, and upon receiving a personal query about the user, a generative artificial intelligence memory agent of the application outputs an answer to the personal query based on the personal data of the user.

18. The system of claim 12, wherein the graphical user interface further comprises a digital next activities button, and upon receiving a selection of the digital next activities button, the assistance application is configured to display a list of upcoming activities associated with the user.

19. The system of claim 12, wherein the assistance application comprises a communication module, and upon receipt of media data having a non-character based format, the communication module performs a binary encoding on the media data to convert the media data into one or more corresponding characters of a character based format, adds the corresponding characters as payload data to one or more MQTT messages and transmits the MQTT messages to a trusted advisor device according to a MQTT protocol.

20. The system of claim 19, wherein upon receipt of an MQTT message from the trusted advisor device, the communication module parses the payload data from the MQTT message, and converts characters of the payload data to corresponding the media data having the non-character based format according to the binary encoding, and further wherein the application outputs the media data on at least one of the touchscreen and the speakers.

21. The system of claim 19, wherein the media data comprises at least one of images, video and audio.

22. The system of claim 19, wherein the binary encoding is Base64 encoding.

23. A method for providing care to one or more users, the method comprising:
providing an assistance device including:
a protective housing;
a touch screen coupled with the housing for displaying images and receiving touch commands;
one or more cameras coupled with the housing;
one or more microphones coupled with the housing;
one or more speakers coupled with the housing;
a finger touch pad coupled with the housing for receiving contact from a finger of a user;
one or more sensors operably coupled with the finger touch pad; and
a non-transitory computer-readable memory storing an assistance application having a graphical user interface that is displayed on the touch screen, wherein the graphical user interface includes one or more digital activity buttons each associated with a desired activity and a time, an image/video window that displays at least one of a video and an image, and a speech/text window that displays text messages received by the device; and
measuring, with the sensors, at least one of a heart rate, a blood-oxygen level and a temperature of the user based on measurements received by the sensors via the finger touch pad while being contacted by the finger of the user.

24. The method of claim 23, further comprising, upon selection of one of the digital activity buttons, publishing a message with the application to one or more trusted advisor devices that are subscribed to the assistance device, the message identifying the activity as being completed.

25. The method of claim 23, further comprising, upon receiving a fullscreen command, changing the image/video window from a first size that is smaller than the touch screen to a larger size that is the same size as the touch screen with the application thereby increasing the size of images or video displayed by the image/video window.

26. The method of claim 23, wherein the graphical user interface includes a weather tile, further comprising generating images and/or text on the touch screen with a generative artificial intelligence weather agent of the assistance application, the images and/or text indicating current suggested clothing and future suggested clothing based on at least one of a current temperature, a current weather, a current humidity and a forecast weather for a location of the assistance device.

27. The method of claim 23, wherein the graphical user interface further comprises a digital call button that is associated with one or more trusted advisor devices, further comprising, upon receiving a call command, initiating a telephone call to at least one of the trusted advisor devices.

28. The method of claim 23, wherein the graphical user interface further comprises a digital assistant button associated with a database of personal data about a life of the user, further comprising, upon receiving a personal query about the user, outputting an answer to the personal query based on the personal data of the user with a generative artificial intelligence memory agent of the application.

29. The method of claim 23, wherein the graphical user interface further comprises a digital next activities button, further comprising, upon receiving a selection of the digital next activities button, displaying a list of upcoming activities associated with the user.

30. The method of claim 23, wherein the assistance application comprises a communication module, further comprising, with the communication module and upon receipt of media data having a non-character based format, performing a binary encoding on the media data to convert the media data into one or more corresponding characters of a character based format, adding the corresponding characters as payload data to one or more MQTT messages and transmitting the MQTT messages to a trusted advisor device according to a MQTT protocol.

31. The method of claim 30, further comprising, with the communication module and upon receipt of an MQTT message from the trusted advisor device, parsing the payload data from the MQTT message, converting characters of the payload data to corresponding the media data having the non-character based format according to the binary encoding, and outputting the media data on at least one of the touchscreen and the speakers with the application.

32. The method of claim 30, wherein the media data comprises at least one of images, video and audio.

33. The method of claim 30, wherein the binary encoding is Base64 encoding.

34. A device for providing a Message Queueing Telemetry Transport (MQTT) communication program to one or more users, the device comprising:
an output interface for outputting first media data from the device;
an input interface for inputting media data having varied non-character-based formats; and
a non-transitory computer-readable memory storing a communication application, wherein upon receipt of the media data having the varied non-character-based formats, the communication application:
standardizes the non-character-based format media data into a character-based format by converting each chunk of the non-character-based format media data to a character of the character-based format;
adds the characters as payload data to one or more MQTT messages; and
transmits the MQTT messages to a second device according to MQTT protocol.

35. The device of claim 34, wherein upon receipt of another MQTT message from the second device, the communication application converts each of the characters of the payload data of the another MQTT message to a corresponding chunk of non-character-based format media data, and outputs the corresponding chunks of the non-character-based format media data via the output interface.

36. The device of claim 34, wherein the non-character-based format media data comprises at least one of images, video and audio.

37. The device of claim 34, wherein the character-based format is ASCII and the converting of each chunk of the non-character-based format media data to the character of the character-based format comprises performing a Base64 encoding the non-character-based format media data.

38. The device of claim 34, wherein the output interface comprises one or more of a display, a touchscreen and speakers.

39. The device of claim 34, wherein the input interface comprises one or more of a camera, a microphone, a touchscreen, a keyboard, a mouse, a graphical user interface and a network interface.

40. A system for providing a Message Queueing Telemetry Transport (MQTT) communication program to one or more users, the system comprising:
a first device including a first output interface for outputting first media content, a first input interface for inputting first non-character-based format media data, and a first non-transitory computer-readable memory storing a first communication application; and
a second device including a second output interface for outputting second media content, a second input interface for inputting second non-character-based format media data, and a second non-transitory computer-readable memory storing a second communication application; wherein upon input of the first non-character-based format media data via the first input interface, the first communication application:
standardizes the first non-character-based format media data into a character-based format by converting each chunk of the first non-character-based format media data to a character of the character-based format;
adds the characters as payload data to one or more first MQTT messages; and
transmits the first MQTT messages to the second device according to MQTT protocol.

41. The system of claim 40, wherein upon receipt of at least one of the first MQTT messages from the first device, the second communication application converts each of the characters of the payload data of the at least one of the MQTT messages to a corresponding chunk of the first non-character-based format media data, and outputs the corresponding chunks of the non-character-based format media data via the second output interface.

42. The system of claim 40, wherein the first non-character-based format media data comprises at least one of images, video and audio.

43. The system of claim 40, wherein the character-based format is ASCII and the converting of each chunk of the first non-character-based format media data to the character of the character-based format comprises performing a Base64 encoding the first non-character-based format media data.

44. The system of claim 40, wherein the first output interface comprises one or more of a display, a touchscreen and speakers.

45. The system of claim 40, wherein the first input interface comprises one or more of a camera, a microphone, a touchscreen, a keyboard, a mouse, a graphical user interface and a network interface.

46. A method for providing a Message Queueing Telemetry Transport (MQTT) communication program to one or more users, the method comprising:
providing an MQTT communication device including an output interface for outputting first media data from the device, an input interface for inputting media data having the varied non-character-based formats and a non-transitory computer-readable memory storing a communication application;
upon receipt of the media data having the varied non-character-based formats by the communication application, standardizing the non-character-based format media data into a character-based format by converting each chunk of the non-character-based format media data to a character of the character-based format;
adding the characters as payload data to one or more MQTT messages with the communication application; and
transmitting the MQTT messages to a second device according to MQTT protocol with the communication application.

47. The method of claim 46, further comprising:
upon receipt of another MQTT message from the second device, converting each of the characters of the payload data of the another MQTT message to a corresponding chunk of non-character-based format media data with the communication application; and
outputting the corresponding chunks of the non-character-based format media data via the output interface with the communication application.

48. The method of claim 46, wherein the non-character-based format media data comprises at least one of images, video and audio.

49. The method of claim 46, wherein the character-based format is ASCII and the converting of each chunk of the non-character-based format media data to the character of the character-based format comprises performing a Base64 encoding the non-character-based format media data.

50. The method of claim 46, wherein the output interface comprises one or more of a display, a touchscreen and speakers.

51. The method of claim 45, wherein the input interface comprises one or more of a camera, a microphone, a touchscreen, a keyboard, a mouse, a graphical user interface and a network interface.
